(19) United States
(12) Patent Application Publication
(10) Pub. No.: US 2020/0245880 A1
(43) Pub. Date: Aug. 6, 2020

(54) HEARTBEAT INDEX DETERMINATION APPARATUS AND METHOD

(71) Applicant: SAMSUNG ELECTRONICS CO., LTD., Suwon-si (KR)

(72) Inventors: Ka Ram CHOI, Hwaseong-si (KR); Seung Keun YOON, Seoul (KR); Sang Kyu KIM, Yongin-si (KR)

(73) Assignee: SAMSUNG ELECTRONICS CO., LTD., Suwon-si (KR)

(21) Appl. No.: 16/740,640

(22) Filed: Jan. 13, 2020

(30) Foreign Application Priority Data
Feb. 1, 2019 (KR) 1020190013830

(51) Int. Cl. A61B 5/024; A61B 5/00

(52) U.S. Cl. CPC A61B 5/02427; A61B 5/02405; A61B 2562/04; A61B 5/7271; A61B 5/7225; A61B 5/7235

(57) ABSTRACT

A heartbeat index determination apparatus may include a pulse wave acquirer configured to acquire a plurality of pulse wave signals that are measured using light of different wavelengths; and a processor configured to measure wavelength-specific heartbeat index values from the plurality of pulse wave signals and determine that a wavelength-specific heartbeat index value that is obtained from the plurality of pulse wave signals a most number of times among the wavelength-specific heartbeat index values is a heartbeat index value of a user.

### CROSS-REFERENCE TO RELATED APPLICATION(S)

This application claims priority from Korean Patent Application No. 10-2019-0013830, filed on Feb. 1, 2019, in the Korean Intellectual Property Office, the disclosure of which is incorporated herein by reference in its entirety.

### BACKGROUND

### 1. Field

Apparatuses and methods consistent with example embodiments relate determining a heartbeat index.

### 2. Description of Related Art

Healthcare technology has attracted much attention due to an aging society and social issues such as increase in medical expenses. Accordingly, not only medical devices that can be utilized by hospitals and inspection agencies but also small-sized medical devices that can be carried by individuals are being developed. In addition, such a small-sized medical device may be worn by a user in the form of a wearable device capable of directly measuring indices related to a heartbeat, such as a heart rate, heart rate variability, and the like, thereby enabling the user to directly measure the heart rate-related indices and monitor the condition of the cardiovascular system.

Therefore, research on miniaturization of a device and a method of accurately determining an index related to a heartbeat using a pulse wave signal has been actively conducted.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Example embodiments provide an apparatus and method for determining a heartbeat index.

According to an aspect of an example embodiment, there is provided a heartbeat index determination apparatus including: a pulse wave acquirer configured to acquire a plurality of pulse wave signals that are measured using light of different wavelengths; and a processor configured to measure wavelength-specific heartbeat index values from the plurality of pulse wave signals and determine that a wavelength-specific heartbeat index value that is obtained from the plurality of pulse wave signals a most number of times among the wavelength-specific heartbeat index values, is a heartbeat index value of a user.

The processor may be further configured to determine a peak-to-peak interval of each of the plurality of pulse wave signals and determine the wavelength-specific heartbeat index values using the peak-to-peak interval of each of the plurality of pulse wave signals.

The processor may be further configured to determine the heartbeat index value of the user based on an agreement of the wavelength-specific heartbeat index values.

The processor may be further configured to extract a predetermined number of pulse wave signals having a signal-to-noise ratio (SNR) greater than a threshold from the plurality of pulse wave signals and apply a voting weight to the wavelength-specific heartbeat index values of the extracted predetermined number of pulse wave signals.

When the processor determines that more than one heartbeat index value are obtained from the plurality of pulse wave signals the most number of times among the wavelength-specific heartbeat index values, the processor may be further configured to extract the predetermined number of pulse wave signals having the SNR greater than the threshold.

The processor may be further configured to remove noise from the plurality of pulse wave signals that is acquired by the pulse wave acquirer.

The heartbeat index value of the user may include at least one of a heart rate (HR), a mean of peak to peak (PP) intervals (mPP), a standard deviation of PP intervals (SDNN), a coefficient variation of PP intervals (CVNN), a square root of a mean of a sum of squares of differences between adjacent PP intervals (RMSSD), a number of pairs of PP intervals with differences more than 20 ms in percentage to all PP intervals (pNN20), a number of pairs of PP intervals with differences more than 50 ms in percentage to all PP intervals (pNN50), power density spectra of a predetermined very low frequency (VLF), power density spectra of a predetermined low frequency band (LF), power density spectra of a predetermined high frequency band (HF), total power density spectra (TF), a ratio between LF and HF (LF/HF), a ratio between HF and TF (HF/TF), normalized VLF (nVLF), normalized LF (nLF), normalized HF (nHF), a difference between LF and HF (dLFHF), a sympathetic modulation index (SMI), a vagal modulation index (VMI), and a sympathovagal balance index (SVI).

The plurality of pulse wave signals may be photoplethysmogram (PPG) signals.

The pulse wave acquirer may be further configured to acquire the plurality of pulse wave signals from an external device.

The pulse wave acquirer may include a plurality of light sources configured to emit the light of different wavelengths to an object of interest of the user and at least one photodetector configured to measure the plurality of pulse wave signals by receiving light returning from the object of interest.

The pulse wave acquirer may include a light source configured to emit light of a predetermined wavelength to an object of interest of the user and a plurality of photodetectors configured to measure the plurality of pulse wave signals by receiving light returning from the object of interest.

Each of the plurality of photodetectors may include a filter configured to filter the light returning from the object of interest.

According to an aspect of another example embodiment, there is provided a method of determining a heartbeat index, including: acquiring a plurality of pulse wave signals that are measured using light of different wavelengths; measuring wavelength-specific heartbeat index values from the plurality of pulse wave signals; and determining that a wavelength-specific heartbeat index value that is obtained from the plurality of pulse wave signals a most number of times among the wavelength-specific heartbeat index values is a heartbeat index value of a user.

The measuring the wavelength-specific heartbeat index values may include determining a peak-to-peak interval of each of the plurality of pulse wave signals and determining the wavelength-specific heartbeat index values using the peak-to-peak interval of each of the plurality of pulse wave signals.

The method may further include determining the heartbeat index value of the user based on an agreement of the wavelength-specific heartbeat index values.

The determining may include extracting a predetermined number of pulse wave signals having a signal-to-noise ratio (SNR) than a threshold from the plurality of pulse wave signals and applying a voting weight to the wavelength-specific heartbeat index values of the extracted predetermined number of pulse wave signals.

The extracting the predetermined number of pulse wave signals may include, in response to determining that there are more than one heartbeat index value that are obtained from the plurality of pulse wave signals the most times among the wavelength-specific heartbeat index values, extracting the predetermined number of pulse wave signals having the SNR greater than the threshold.

The method may further include removing nose from the plurality of pulse wave signals.

The heartbeat index value of the user comprises includes at least one of a heart rate (HR), a mean of peak to peak (PP) intervals (mPP), a standard deviation of PP intervals (SDNN), a coefficient variation of PP intervals (CVNN), a square root of a mean of a sum of squares of differences between adjacent PP intervals (RMSSD), a number of pairs of PP intervals with differences more than 20 ms in percentage to all PP intervals (pNN20), a number of pairs of PP intervals with differences more than 50 ms in percentage to all PP intervals (pNN50), power density spectra of a predetermined very low frequency (VLF), power density spectra of a predetermined low frequency band (LF), power density spectra of a predetermined high frequency band (HF), total power density spectra (TF), a ratio between LF and HF (LF/HF), a ratio between HF and TF (HF/TF), normalized VLF (nVLF), normalized LF (nLF), normalized HF (nHF), a difference between LF and HF (dLFHF), a sympathetic modulation index (SMI), a vagal modulation index (VMI), and a sympathovagal balance index (SVI).

The plurality of pulse wave signals may be photoplethysmogram (PPG) signals.

### BRIEF DESCRIPTION OF THE DRAWINGS

The above and/or other aspects will be more apparent by describing certain example embodiments, with reference to the accompanying drawings, in which:

FIG. 1 is a diagram illustrating a heartbeat index determination apparatus according to an example embodiment;

FIGS. 2A, 2B, and 2C are diagrams illustrating example embodiments of a pulse wave acquirer;

FIG. 3 is a graph for describing peak-to-peak intervals;

FIG. 4 is a diagram illustrating a heartbeat index determination apparatus according to another example embodiment;

FIG. 5 is a flowchart illustrating a method of determining a heartbeat index according to an example embodiment;

FIG. 6 is a flowchart illustrating a method of determining a heartbeat index according to another example embodiment; and

FIG. 7 is a perspective view of a wrist wearable device.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

The following detailed description is provided to assist the reader in gaining a comprehensive understanding of the methods, apparatuses and/or systems described herein. Various changes, modifications, and equivalents of the systems, apparatuses and/or methods described herein will suggest themselves to those of ordinary skill in the art. In the following description, a detailed description of known functions and configurations incorporated herein will be omitted when it may obscure the subject matter with unnecessary detail. Throughout the drawings and the detailed description, unless otherwise described, the same drawing reference numerals will be understood to refer to the same elements, features, and structures. The relative size and depiction of these elements may be exaggerated for clarity, illustration, and convenience.

As used herein, the singular forms are intended to include the plural forms as well, unless the context clearly indicates otherwise. It will be further understood that the terms “comprises” and/or “comprising,” or “includes” and/or “including” when used in this description, specify the presence of stated features, numbers, steps, operations, elements, components or combinations thereof, but do not preclude the presence or addition of one or more other features, numbers, steps, operations, elements, components or combinations thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

It will also be understood that the elements or components in the following description are discriminated in accordance with their respective main functions. In other words, two or more elements may be made into one element or one element may be divided into two or more elements in accordance with a subdivided function. Additionally, each of the elements in the following description may perform a part or whole of the function of another element as well as its main function, and some of the main functions of each of the elements may be performed exclusively by other elements. Each element may be realized in the form of a hardware component, a software component, and/or a combination thereof.

A heartbeat index determination apparatus as will be described below may be implemented as a software module or in the form of a hardware chip and may be mounted in an electronic device. In this case, the electronic device may include a mobile phone, a smartphone, a tablet computer, a notebook computer, a personal digital assistant (PDA), a portable multimedia player (PMP), a navigation device, an MP3 player, a digital camera, a wearable device, and the like, and the wearable device may include a wrist watch type, a wrist band type, a ring type, a belt type, a necklace type, an ankle band type, a thigh band type, a forearm band type, and the like. However, the electronic device and the wearable device are not limited to the above examples.

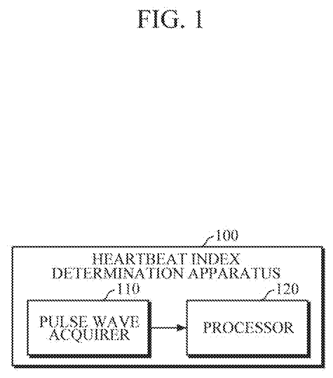

FIG. 1 is a diagram illustrating a heartbeat index determination apparatus according to an example embodiment.

Referring to FIG. 1, the heartbeat index determination apparatus 100 may include a pulse wave acquirer 110 and a processor 120.

The pulse wave acquirer 110 may acquire a plurality of pulse wave signals of an object of interest. The plurality of pulse wave signals may be photoplethysmogram (PPG) signals that are measured using different wavelengths of light (e.g., a blue band, a green band, a red band, an infrared band, etc.). Here, the object of interest may be a body part, and examples of the object of interest include a peripheral region of the human body, such as a finger, a toe, or the like, or an upper wrist region which is a region of the surface of the wrist adjacent to a radial artery and where capillary blood or venous blood passes.

According to one embodiment, the pulse wave acquirer 110 may receive the pulse wave signals of the object of interest from an external device that measures and/or stores the pulse wave signals. In this case, the pulse wave acquirer 110 may use various communication technologies, such as Bluetooth communication, Bluetooth low energy (BLE) communication, near field communication (NFC), wireless local area network (WLAN) communication, ZigBee communication, infrared data association (IrDA) communication, Wi-Fi direct (WFD) communication, ultra-wideband (UWB) communication, Ant+ communication, Wi-Fi communication, RFID communication, 3G communication, 4G communication, 5G communication, and the like.

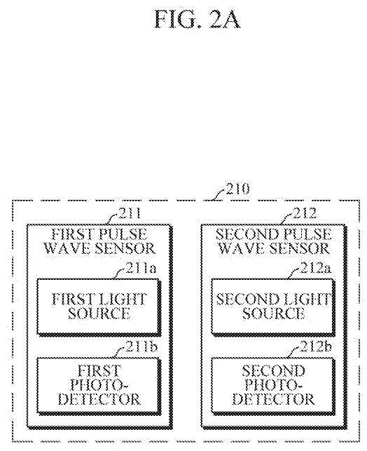

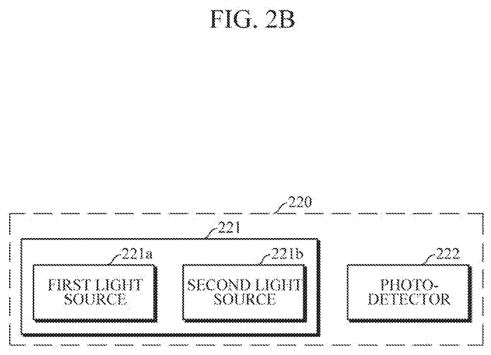

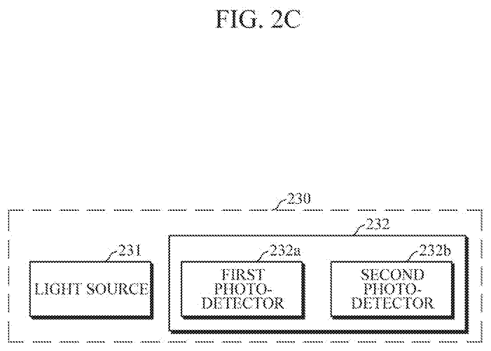

According to another embodiment, the pulse wave acquirer 110 may emit light to the object of interest and measure a pulse wave signal by receiving light reflected or scattered from the object of interest, which will be described in detail below with reference to FIGS. 2A to 2C.

The processor 120 may control overall operation of the heartbeat index determination apparatus 100 and may be configured with one or more processors, a memory, or a combination thereof.

The processor 120 may control the pulse wave acquirer 110 to acquire a plurality of pulse wave signals and may determine a heartbeat index of the object of interest based on the plurality of pulse wave signals. For example, when the pulse wave acquirer 110 is implemented as a communication interface to receive the pulse wave signals of the object of interest from an external device, the processor 120 may send a request for the plurality of pulse wave signals to the external device through the pulse wave acquirer 110. In another example, when the pulse wave acquirer 110 is implemented as an optical sensor or a spectrometer to measure the plurality of pulse wave signals, the processor 120 may drive the pulse wave acquirer 110 to measure the plurality of pulse wave signals according to a predetermined driving condition. In this case, the driving condition may include an emission time of a light source, a driving order of light sources, and current intensity applied to a light source, pulse duration, and the like.

The processor 120 may remove noise from the plurality of pulse wave signals that are obtained by the pulse wave acquirer 110. For example, the processor 120 may remove noise from the plurality of pulse wave signals using various filtering methods, such as a band-pass filter, a moving average filter, and the like.

The processor 120 may analyze the plurality of pulse wave signals and determine a heartbeat index value (hereinafter referred to as a “wavelength-specific heartbeat index”) for each pulse wave signal or for each wavelength. According to one embodiment, the heartbeat index may include at least one of a heart rate (HR), a mean of peak to peak (PP) intervals (mPP), a standard deviation of PP intervals (SDNN), a coefficient variation of PP intervals (CVNN), a square root of the mean of the sum of the squares of differences between adjacent PP intervals (RMSSD), the number of pairs of PP intervals with differences more than 20 ms in percentage to all PP intervals (pNN20), the number of pairs of PP intervals with differences more than 50 ms in percentage to all PP intervals (pNN50), power density spectra of a predetermined very low frequency (VLF), power density spectra of a predetermined low frequency band (LF), power density spectra of a predetermined high frequency band (HF), total power density spectra (TF), a ratio between LF and HF (LF/HF), a ratio between HF and TF (HF/TF), normalized VLF (nVLF), normalized LF (nLF), normalized HF (nHF), a difference between LF and HF (dLFHF), a sympathetic modulation index (SMI), a vagal modulation index (VMI), and a sympathovagal balance index (SVI), as shown in Tables 1 and 2 below.

TABLE 1
Examples of heartbeat index in time domain

| Heartbeat | |
|---|---|
| Index | Description |
| HR | Heart rate |
| mPP | Mean of peak to peak (PP) intervals |
| SDNN | Standard deviation of PP intervals |
| CVNN | Coefficient variation of PP intervals |
| RMSSD | Square root of the mean of the sum of the squares |
| | of differences between adjacent PP intervals |
| pNN20 | Number of pairs of PP intervals with differences |
| | more than 20 ms in percentage to all PP intervals |
| pNN50 | Number of pairs of PP intervals with differences |
| | more than 50 ms in percentage to all PP intervals |

TABLE 2
Examples of heartbeat index in frequency domain

| Heartbeat | |
|---|---|
| Index | Description |
| VLF | Power density spectra of very low frequency |
| | (0.00 Hz - 0.04 Hz) |
| LF | Power density spectra of low frequency band |
| | (0.04 Hz - 0.15 Hz) |
| HF | Power density spectra of high frequency band |
| | (0.15 Hz - 0.4 Hz) |
| TF | Total power density spectra |
| LF/HF | Ratio between LF and HF |
| LF/TF | Ratio between LF and TF |
| HF/TF | Ratio between HF and TF |
| nVLF | Normalized VLF |
| nLF | Normalized LF |
| nHF | Normalized HF |
| dLFHF | Difference between LF and HF |
| SMI | Sympathetic modulation index |
| VMI | Vagal modulation index |
| SVI | Sympathovagal balance index |

For example, the processor 120 may detect peak points or foot points from each of the pulse wave signals and determine a peak-to-peak interval based on the detected peak points or foot points. In addition, the processor 120 may determine the wavelength-specific heartbeat index values using the peak-to-peak interval detected from each of the pulse wave signals.

The processor 120 may determine that a heartbeat index value that appears the most times among the determined wavelength-specific heartbeat index values is a final heartbeat index value. The final heartbeat index value may be provide to a user as the user's heartbeat index value. For example, it is assumed that a heartbeat index value PR_{λ1} determined by analyzing a pulse wave signal PPG₁ that has been measured using light of wavelength λ₁ is 68, a heartbeat index value PR_{λ2} determined by analyzing a pulse wave signal PPG₂ that has been measured using light of wavelength λ₂ is 64, a heartbeat index value PR_{λ3} determined by analyzing a pulse wave signal PPG₃ that has been measured using light of wavelength λ₃ is 66 and a heartbeat index value PR_{λ4} determined by analyzing a pulse wave signal PPG₄ that has been measured using light of wavelength λ₄ is 66. In this case, the heartbeat index value of 68 and the heartbeat index value of 64 appear only once but the heartbeat index value of 66 appears twice, and hence the processor 120 may determine the heartbeat index value of 66, which appears the most times among the heartbeat index values, as the final heartbeat index value.

The processor 120 may determine the reliability of the final heartbeat index value based on the agreement of the wavelength-specific index values. In the above example, the ratio among the heartbeat index values (68, 64, and 66) is 1:1:2. In this case, the processor 120 may determine that the reliability of the final heartbeat index value, 66, is (2*100)/4=50%.

According to an example embodiment, the processor 120 may determine a signal-to-noise ratio (SNR) for each of the plurality of pulse wave signals and apply a voting weight to the wavelength-specific heartbeat index value that corresponds to the pulse wave signal having a high SNR. For example, when there are a plurality of heartbeat index values that appear the most times among the wavelength-specific heartbeat index values, the processor 120 may extract a predetermined number of pulse wave signals having a high SNR or extract a predetermined number of pulse wave signals having an SNR greater than a predetermined threshold, and may apply a voting weight to the wavelength-specific heartbeat index value corresponding to each of the extracted pulse wave signals to determine a final heartbeat index value. In this case, the processor 120 may apply a higher voting weight to the pulse wave signal having a higher SNR. For example, it is assumed that a heartbeat index value PR_{λ1} determined by analyzing a pulse wave signal PPG₁ that has been measured using light of wavelength λ₁ is 68, a heartbeat index value PR_{λ2} determined by analyzing a pulse wave signal PPG₂ that has been measured using light of wavelength λ₂ is 68, a heartbeat index value PR_{λ3} determined by analyzing a pulse wave signal PPG₃ that has been measured using light of wavelength λ₃ is 66 and a heartbeat index value PR_{λ4} determined by analyzing a pulse wave signal PPG₄ that has been measured using light of wavelength λ₄ is 66. In addition, it is assumed that an SNR of the pulse wave signal PPG₁ is 50 dB, an SNR of the pulse wave signal PPG₂ is 60 dB, an SNR of the pulse wave signal PPG₃ is 55 dB and an SNR of the pulse wave signal PPG₄ is 65 dB. The processor 120 may extract two pulse wave signals PPG₂ and PPG₄ that have relatively higher SNRs to give the extracted two pulse wave signals PPG₂ and PPG₄ higher voting weights than a default voting weight (e.g., 1). For example, the processor 120 may apply a first voting weight of 2 that is greater than 1 to the heartbeat index value PR_{λ2} corresponding to the pulse wave signal PPG₂ and apply a second voting weight of 3 that is greater than the first voting weight to the heartbeat index value PR_{λ4} corresponding to the pulse wave signal PPG₄. As a result, the processor 120 may assign voting weights of 1, 2, 1, and 3 to the pulse wave signals PPG₁-PPG₄, respectively, as shown in a table below. The processor 120 may add the voting weights of 1 and 2 of the pulse wave signals PPG₁ and PPG₂ that have the same heartbeat index value of 68, and may obtain a first resulting value of 3 (i.e., 1+(2*1)=3). The processor 120 may add the voting weights of 1 and 3 of the pulse wave signals PPG₃ and PPG₄ that have the same heartbeat index value of 66 (i.e., 1+(3*1)=4), and may obtain a second resulting value of 4. The processor 120 may compare the first resulting value of 3 for the heart index value of 68 with the second resulting value of 4 for the heart index value of 66, and may determine the heartbeat index value of 66 as a final heartbeat index value because the first resulting value of 4 for the heart index value of 66 is greater than the second resulting value of 3 for the heart index value of 68.

| Pulse Wave | Heartbeat | Signal-to-Noise | Voting |
|---|---|---|---|
| Signal | Index Value | Ratio | Weight |
| PPG₁ | PR_{λ1} = 68 | 50 | 1 |
| PPG₂ | PR_{λ2} = 68 | 60 | 2 |
| PPG₃ | PR_{λ3} = 66 | 55 | 1 |
| PPG₄ | PR_{λ4} = 66 | 65 | 3 |

The processor 120 may predict cardiovascular information and/or cardiovascular disease risk using the final heartbeat index value. In this case, the cardiovascular information may include blood pressure, vascular age, heart age, a degree of arteriosclerosis, cardiac output, a vascular compliance, blood glucose, blood triglycerides, peripheral vascular resistance, and the like. The cardiovascular disease may include arrhythmia, diabetes, sympathetic/parasympathetic dysfunction, and sleep apnea. In this case, the processor 120 may use a cardiovascular information estimation model that defines the relationships between heartbeat index values and cardiovascular information and/or a disease risk estimation model that defines relationships between heartbeat index values and cardiovascular diseases.

FIGS. 2A, 2B, and 2C are diagrams illustrating example embodiments of a pulse wave acquirer. The embodiments shown in FIGS. 2A, 2B, and 2C may be embodiments of the pulse wave acquirer 110 of FIG. 1. Hereinafter, various example embodiments of the pulse wave acquirer that measures a plurality of pulse wave signals using light of different wavelengths will be described with reference to FIGS. 2A, 2B, and 2C.

Referring to FIG. 2A, a pulse wave acquirer 510 according to an example embodiment may be formed as an array of pulse wave sensors for measuring a plurality of pulse wave signals using light of different wavelengths. As shown in FIG. 2A, the pulse wave acquirer 210 may include a first pulse wave sensor 211 and a second pulse wave sensor 212. However, the first and second pulse wave sensors are provided for convenience of description, and the number of pulse wave sensors forming the pulse wave sensor array is not particularly limited.

The first pulse wave sensor 211 may include a first light source 211a configured to emit light of a first wavelength to an object of interest. In addition, the first pulse wave sensor 211 may include a first photodetector 211b configured to measure a first pulse wave signal by receiving light of the first wavelength returning from the object of interest irradiated by the first light source 211a.

The second pulse wave sensor 212 may include a second light source 212a configured to emit light of a second wavelength to the object of interest. In addition, the second pulse wave sensor 212 may include a second photodetector 212b configured to measure a second pulse wave signal by receiving light of the second wavelength returning from the object of interest irradiated by the second light source 212a. In this case, the first wavelength and the second wavelength may differ from each other.

In this case, the first light source 211a and the second light source 212a may include a light emitting diode, a laser diode, and a phosphor, but are not limited thereto. In addition, the first photodetector 211b and the second photodetector 212b may include a photodiode, a photo transistor PTr, or a charge-coupled device (CCD), but are not limited thereto.

Referring to FIG. 2B, a pulse wave acquirer 220 according to another example embodiment may include a light source portion 221 including a plurality of light sources 221a and 221b and a photodetector 222. However, although FIG. 2B illustrates two light sources in the light source portion 221, this is merely for convenience of description, and the number of light sources is not particularly limited.

The first light source 221a may emit light of a first wavelength to an object of interest and the second light source 221b may emit light of a second wavelength to the object of interest. In this case, the first wavelength and the second wavelength may differ from each other.

The photodetector 222 may receive light of different wavelengths returning from the object of interest and measure a plurality of pulse wave signals.

For example, the first light source 221a and the second light source 221b may be driven in a time-division manner according to the control of the processor and sequentially emit light to the object of interest. In this case, a driving condition, such as emission times of the first and second light sources, a driving order of the first and second light sources, and current intensity, pulse duration, and the like, may be set in advance. The processor may control the driving of each light source 221a and 221b by referring to the light source driving condition.

The photodetector 222 may measure a first pulse wave signal and a second pulse wave signal by sequentially detecting the light of the first wavelength and the light of the second wavelength returning from the object of interest which has been sequentially irradiated by the first light source 221a and the second light source 221b.

Referring to FIG. 2C, a pulse wave acquirer 230 according to another example embodiment may include a light source 231 and a photodetector portion 232. The photodetector portion 232 may include a first photodetector 232a and a second photodetector 232. However, although FIG. 2C illustrates two photodetectors in the photodetector portion 231, this is merely for convenience of description, and the number of photodetectors is not particularly limited.

The light source 231 may emit light of a predetermined wavelength band to an object of interest. In this case, the single light source 231 may be configured to emit light of a wide wavelength band including visible light and/or infrared light.

The photodetector portion 232 may measure a plurality of pulse wave signals by receiving light of the predetermined wavelength band returning from the object of interest. To this end, the photodetector portion 232 may be configured to have a plurality of different response characteristics.

For example, the first photodetector 232a and the second photodetector 232b may be configured with photodiodes having different measurement ranges such that the first photodetector 232a and the second photodetector 232b respond to light of different wavelengths returning from the object of interest. Alternatively, either of the first photodetector 232a or the second photodetector 232b may have a color filter on a front surface thereof so as to receive light of different wavelengths, or both of the two photodetectors 232a and 232b may have different color filters on their front surfaces. Alternatively, the first photodetector 232a and the second photodetector 232b may be disposed at different distances to the light source 231. In this case, the photodetector that is disposed at a relatively close distance to the light source 231 may detect light of a short wavelength band and the photodetector that is disposed at a relatively far distance to the light source 231 may detect light of a long wavelength band.

The embodiments of the pulse wave acquirer for measuring a plurality of pulse wave signals of different wavelengths have been described with reference to FIGS. 2A to 2C. However, these embodiments are merely examples, and the pulse wave acquirer is not limited thereto. The number and arrangement of light sources and photodetectors may vary according to the purpose of use of the pulse wave acquirer and the size and shape of an electronic device in which the pulse wave acquirer is mounted.

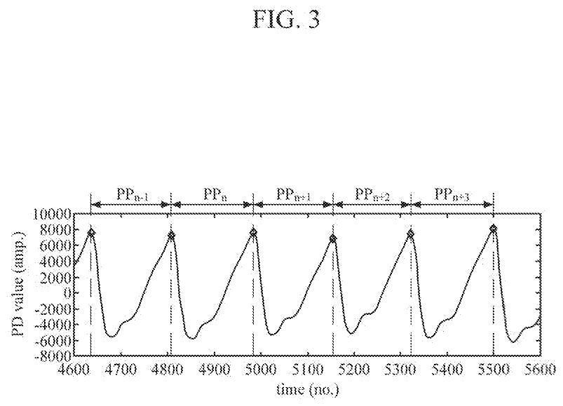

FIG. 3 is a graph for describing peak-to-peak intervals.

Referring to FIG. 3, a processor 120 may determine peak points of a pulse wave signal and detect time intervals ( . . . , PPₙ₋₁, PPₙ, PPₙ₊₁, PPₙ₊₂, PPₙ₊₃, and so on) between adjacent peak points as peak-to-peak intervals.

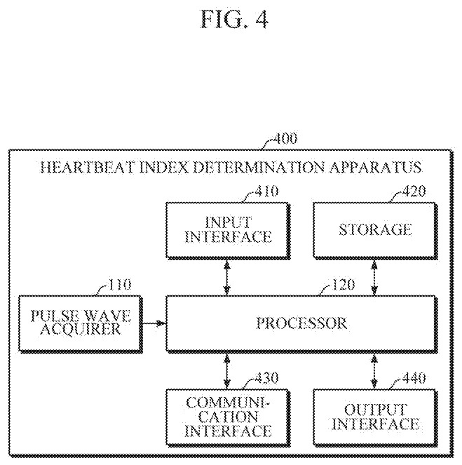

FIG. 4 is a diagram illustrating a heartbeat index determination apparatus according to another example embodiment.

Referring to FIG. 4, the heartbeat index determination apparatus 400 may include a pulse wave acquirer 110, a processor 120, an input interface 410, a storage 420, a communication interface 430, and an output interface 440. Here, the pulse wave acquirer 110 and the processor 120 may be substantially the same as those described with reference to FIGS. 1 to 3, and thus detailed descriptions thereof will not be reiterated.

The input interface 410 may receive various operation signals from a user. According to one embodiment, the input interface 410 may include a key pad, a dome switch, a touch pad (resistive/capacitive), a jog wheel, a jog switch, a hardware button, and the like. In particular, when a touchpad has a layered structure with a display, this structure may be referred to as a touch screen.

A program or commands for operation of the heartbeat index determination apparatus 400 may be stored in the storage 420, and input data, processed data and output data of the heartbeat index determination apparatus 400, and data required for data processing in the heartbeat index determination apparatus 400 may be stored in the storage 420. The storage 420 may include a storage medium of at least one type of flash memory type, hard disk type, multimedia card micro type, card-type memory (e.g., secure digital (SD) or extreme digital (XD) memory), random access memory (RAM), static random access memory (SRAM), read only memory (ROM), electrically erasable programmable read only memory (EEPROM), programmable read only memory (PROM), magnetic memory, magnetic disk, optical disk, and the like. In addition, the heartbeat index determination apparatus 400 may operate an external storage medium, such as web storage providing a storage function of the storage 420 on the Internet.

The communication interface 430 may communicate with an external device. For example, the communication interface 430 may transmit the input data, stored data stored, and processed data of the heartbeat index determination apparatus 400 to the external device, or may receive a variety of data useful to estimate cardiovascular information from the external device.

In this case, the external device may be a medical device that uses the input data, stored data, and processed data of the heartbeat index determination apparatus 400 or a printer or a display device for outputting results. In addition, the external device may be a digital TV, a desktop computer, a mobile phone, a smartphone, a tablet computer, a notebook computer, a personal digital assistant (PDA), a portable multimedia player (PMP), a navigation system, an MP3 player, a digital camera, a wearable device, or the like, but is not limited thereto.

The communication interface 430 may communicate with the external device using Bluetooth, BLE, NFC, WLAN communication, ZigBee communication, IrDA communication, WFD communication, UWB communication, Ant+ communication, Wi-Fi communication, RFID communication, 3G communication, 4G communication, 5G communication, and the like. However, these are merely examples, and the embodiment is not limited thereto.

The output interface 440 may output the input data, stored data, and processed data of the heartbeat index determination apparatus 400. According to one embodiment, the output interface 440 may output the input data, stored data, and processed data of the heartbeat index determination apparatus 400 in at least one of an audible method, a visual method, and a tactile method. To this end, the output interface 440 may include a display, a speaker, a vibrator, and the like.

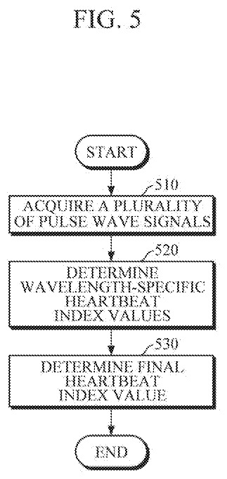

FIG. 5 is a flowchart illustrating a method of determining a heartbeat index according to an example embodiment. The method shown in FIG. 5 may be performed by the heartbeat index determination apparatus 100 or 400 of FIG. 1 or 4.

Referring to FIG. 5, the heartbeat index determination apparatus may acquire a plurality of pulse wave signals of an object of interest in operation 510. The pulse wave signal may be a PPG signal, and the plurality of pulse wave signals may be pulse wave signals measured using different wavelengths of light (e.g., a blue band, a green band, a red band, an infrared band, etc.).

According to one embodiment, the heartbeat index determination apparatus may receive pulse wave signals of the object of interest from an external device, which measures and/or stores the pulse wave signals, using the various communication technologies described above.

According to another embodiment, the heartbeat index determination apparatus may emit light of different wavelengths to the object of interest and measure the pulse wave signals by receiving the light reflected or scattered from the object of interest.

The heartbeat index determination apparatus may determine wavelength-specific heartbeat index values by analyzing the plurality of pulse wave signals in operation 520. According to one embodiment, the heartbeat index may include at least one of HR, mPP, SDNN, CVNN, RMSSD, pNN20, pNN50, VLF, LF, HF, TF, LF/HF, LF/TF, HF/TF, nVLF, nLF, nHF, dLFHF, SMI, VMI, and SVI, as shown in Tables 1 and 2 above.

For example, the heartbeat index determination apparatus may detect peak points or foot points from each of the pulse wave signals and determine a peak-to-peak interval based on the detected peak points or foot points. In addition, the heartbeat index determination apparatus may determine the wavelength-specific heartbeat index values using the peak-to-peak interval detected from each of the pulse wave signals.

The heartbeat index determination apparatus may determine a heartbeat index value that appears the most times among the determined wavelength-specific heartbeat index values is a final heartbeat index value in operation 530.

According to one embodiment, the heartbeat index determination apparatus may determine an SNR for each of the plurality of pulse wave signals and apply a voting weight to the wavelength-specific heartbeat index value that corresponds to the pulse wave signal having a high SNR. For example, when there are a plurality of heartbeat index values that appear the most times among the wavelength-specific heartbeat index values, the heartbeat index determination apparatus may extract a predetermined number of pulse wave signals having a high SNR or extract a predetermined number of pulse wave signals having an SNR greater than a predetermined threshold, and may apply a voting weight to the wavelength-specific heartbeat index value corresponding to each of the extracted pulse wave signals to determine a final heartbeat index value. In this case, the heartbeat index determination apparatus may apply a higher voting weight to the pulse wave signal having a higher SNR.

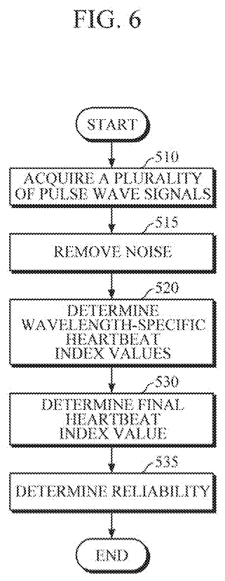

FIG. 6 is a flowchart illustrating a method of determining a heartbeat index according to another example embodiment. The method of FIG. 6 may be performed by the heartbeat index determination apparatus 100 or 400 of FIG. 1 or 4. Operations 510, 520, and 530 of FIG. 6 are substantially the same as the operations described with reference to FIG. 5, and thus detailed descriptions thereof will not be reiterated.

Referring to FIG. 6, the heartbeat index determination apparatus may remove noise from a plurality of acquired pulse wave signals in operation 515. For example, the heartbeat index determination apparatus may remove noise from the plurality of acquired pulse wave signals using various filtering methods, such as a band-pass filter, a moving average filter, and the like.

The heartbeat index determination apparatus may determine the reliability of the final heartbeat index value based on the agreement of the wavelength-specific heartbeat index values in operation 535.

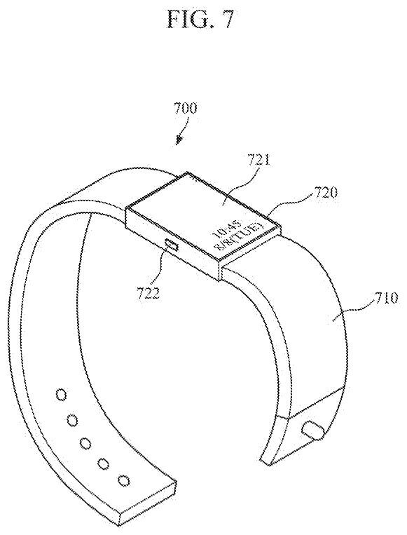

FIG. 7 is a perspective view of a wrist wearable device.

Referring to FIG. 7, the wrist wearable device 700 may include a strap 710 and a main body 720.

The strip 710 may be composed of a plurality of strip members, each of which is configured to be bent in a form that wraps around a wrist of a user. However, this is merely one embodiment and the present embodiment is not limited thereto. That is, the strap 710 may be configured in the form of a flexible band.

The above-described heartbeat index determination apparatus 100 or 400 may be mounted inside the main body 720. In addition, a battery for supplying power to the wrist wearable device 700 and the heartbeat index determination apparatus 100 or 400 may be embedded in the main body 720.

A pulse wave sensor may be mounted in a lower part of the main body 720 so as to be exposed to the wrist of the user. Accordingly, when the user wears the wrist wearable device 700, the pulse wave sensor may be naturally brought into contact with the user's skin. In this case, the pulse wave sensor may emit light to an object of interest and measure a pulse wave of the object of interest by receiving light reflected or scattered from the object of interest.

The wrist wearable device 700 may further include an input interface 721 and a display 722, which are mounted in the main body 720. The input interface 721 may receive various operation signals from the user. The display 722 may display data processed by the wrist wearable device 700 and the heartbeat index determination apparatus 100 or 400, processing result data, and the like.

The current embodiments can be implemented as computer readable codes in a computer readable record medium. Codes and code segments constituting the computer program can be easily inferred by a skilled computer programmer in the art. The computer readable record medium includes all types of record media in which computer readable data are stored. Examples of the computer readable record medium include a ROM, a RAM, a CD-ROM, a magnetic tape, a floppy disk, and an optical data storage. Further, the record medium may be implemented in the form of a carrier wave such as Internet transmission. In addition, the computer readable record medium may be distributed to computer systems over a network, in which computer readable codes may be stored and executed in a distributed manner.

A number of example embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A heartbeat index determination apparatus comprising:
a pulse wave acquirer configured to acquire a plurality of pulse wave signals that are measured using light of different wavelengths; and
a processor configured to:
measure wavelength-specific heartbeat index values from the plurality of pulse wave signals, and
determine that a wavelength-specific heartbeat index value that is obtained from the plurality of pulse wave signals a most number of times among the wavelength-specific heartbeat index values is a heartbeat index value of a user.

2. The heartbeat index determination apparatus of claim 1, wherein the processor is further configured to:
determine a peak-to-peak interval of each of the plurality of pulse wave signals, and
determine the wavelength-specific heartbeat index values using the peak-to-peak interval of each of the plurality of pulse wave signals.

3. The heartbeat index determination apparatus of claim 1, wherein the processor is further configured to determine the heartbeat index value of the user based on an agreement of the wavelength-specific heartbeat index values.

4. The heartbeat index determination apparatus of claim 1, wherein the processor is further configured to:
extract a number of pulse wave signals having a signal-to-noise ratio (SNR) greater than a threshold from the plurality of pulse wave signals; and
apply a voting weight to the wavelength-specific heartbeat index values of the extracted number of pulse wave signals.

5. The heartbeat index determination apparatus of claim 4, wherein when the processor is further configured to:
determine that more than one heartbeat index value are obtained from the plurality of pulse wave signals the most number of times among the wavelength-specific heartbeat index values, and
extract the number of pulse wave signals having the SNR greater than the threshold.

6. The heartbeat index determination apparatus of claim 1, wherein the processor is further configured to remove noise from the plurality of pulse wave signals that is acquired by the pulse wave acquirer.

7. The heartbeat index determination apparatus of claim 1, wherein the heartbeat index value of the user comprises at least one of a heart rate (HR), a mean of peak to peak (PP) intervals (mPP), a standard deviation of PP intervals (SDNN), a coefficient variation of PP intervals (CVNN), a square root of a mean of a sum of squares of differences between adjacent PP intervals (RMSSD), a number of pairs of PP intervals with differences more than 20 ms in percentage to all PP intervals (pNN20), a number of pairs of PP intervals with differences more than 50 ms in percentage to all PP intervals (pNN50), power density spectra of a predetermined very low frequency (VLF), power density spectra of a predetermined low frequency band (LF), power density spectra of a predetermined high frequency band (HF), total power density spectra (TF), a ratio between LF and HF (LF/HF), a ratio between HF and TF (HF/TF), normalized VLF (nVLF), normalized LF (nLF), normalized HF (nHF), a difference between LF and HF (dLFHF), a sympathetic modulation index (SMI), a vagal modulation index (VMI), and a sympathovagal balance index (SVI).

8. The heartbeat index determination apparatus of claim 1, wherein the plurality of pulse wave signals are photoplethysmogram (PPG) signals.

9. The heartbeat index determination apparatus of claim 1, wherein the pulse wave acquirer is further configured to acquire the plurality of pulse wave signals from an external device.

10. The heartbeat index determination apparatus of claim 1, wherein the pulse wave acquirer comprises:
a plurality of light sources configured to emit the light of different wavelengths to an object of interest of the user; and
at least one photodetector configured to measure the plurality of pulse wave signals by receiving light returning from the object of interest.

11. The heartbeat index determination apparatus of claim 1, wherein the pulse wave acquirer comprises:
a light source configured to emit light of a predetermined wavelength to an object of interest of the user; and
a plurality of photodetectors configured to measure the plurality of pulse wave signals by receiving light returning from the object of interest.

12. The heartbeat index determination apparatus of claim 11, wherein each of the plurality of photodetectors comprises a filter configured to filter the light returning from the object of interest.

13. A method of determining a heartbeat index, the method comprising:
acquiring a plurality of pulse wave signals that are measured using light of different wavelengths;
measuring wavelength-specific heartbeat index values from the plurality of pulse wave signals; and
determining that a wavelength-specific heartbeat index value that is obtained from the plurality of pulse wave signals a most number of times among the wavelength-specific heartbeat index values is a heartbeat index value of a user.

14. The method of claim 13, wherein the measuring the wavelength-specific heartbeat index values comprises:
determining a peak-to-peak interval of each of the plurality of pulse wave signals; and
determining the wavelength-specific heartbeat index values using the peak-to-peak interval of each of the plurality of pulse wave signals.

15. The method of claim 13, further comprising determining the heartbeat index value of the user based on an agreement of the wavelength-specific heartbeat index values.

16. The method of claim 13, wherein the determining comprises:
extracting a number of pulse wave signals having a signal-to-noise ratio (SNR) than a threshold from the plurality of pulse wave signals; and
applying a voting weight to the wavelength-specific heartbeat index values of the extracted number of pulse wave signals.

17. The method of claim 16, wherein the extracting the number of pulse wave signals comprises, in response to determining that there are more than one heartbeat index value that are obtained from the plurality of pulse wave signals the most number of times among the wavelength-specific heartbeat index values, extracting the number of pulse wave signals having the SNR greater than the threshold.

18. The method of claim 13, further comprising removing nose from the plurality of pulse wave signals.

19. The method of claim 13, wherein the heartbeat index value of the user comprises at least one of a heart rate (HR), a mean of peak to peak (PP) intervals (mPP), a standard deviation of PP intervals (SDNN), a coefficient variation of PP intervals (CVNN), a square root of a mean of a sum of squares of differences between adjacent PP intervals (RMSSD), a number of pairs of PP intervals with differences more than 20 ms in percentage to all PP intervals (pNN20), a number of pairs of PP intervals with differences more than 50 ms in percentage to all PP intervals (pNN50), power density spectra of a predetermined very low frequency (VLF), power density spectra of a predetermined low frequency band (LF), power density spectra of a predetermined high frequency band (HF), total power density spectra (TF), a ratio between LF and HF (LF/HF), a ratio between HF and TF (HF/TF), normalized VLF (nVLF), normalized LF (nLF), normalized HF (nHF), a difference between LF and HF (dLFHF), a sympathetic modulation index (SMI), a vagal modulation index (VMI), and a sympathovagal balance index (SVI).

20. The method of claim 13, wherein the plurality of pulse wave signals are photoplethysmogram (PPG) signals.
